(19) United States
(12) Patent Application Publication
(10) Pub. No.: US 2026/0263386 A1
(43) Pub. Date: Sep. 10, 2026

(54) Treatment of Inflammation in the Central Nervous System

(71) Applicant: Jacek M. Kwiecien, Hamilton (CA)

(72) Inventor: Jacek M. Kwiecien, Hamilton (CA)

(21) Appl. No.: 19/665,765

(22) Filed: May 1, 2026

Related U.S. Application Data
(63) Continuation-in-part of application No. 17/346,726, filed on Jun. 14, 2021, now Pat. No. 12,636,261.

(51) Int. Cl. A61K 31/12; A61P 25/00; A61P 29/00

(52) U.S. Cl. CPC A61K 31/12; A61P 25/00; A61P 29/00

(57) ABSTRACT

The present invention pertains to the anti-inflammatory therapeutic effect of xantohumol in spinal cord injury (SCI). The continuous administration of xanthohumol for 1-8 weeks to SCI rats resulted in improved results in 4 clinical tests used and in lowering and faster elimination of macrophages from the SCI lesion. Since the infiltration of the SCI lesion by numerous phagocytic macrophages indicates a severe destructive inflammation of extraordinary longevity, administration of xanthohumol is neuroprotective and resulted in a better and faster recovery of the locomotor function and the strength and sensory function in the hind limbs, in a shorter period of paralysis of the urinary bladder and in the recovery of the body weight lost due to the SCI surgery. Since the traumatic brain injury (TBI) involving the white matter, and stroke involving the white matter initiate the severe destructive inflammation as in the SCI, the administration of xanthohumol is expected to be anti-inflammatory and neuroprotective in both brain diseases. Since neurodegenerative diseases including Alzheimer's disease, frontotemporal dementia and Parkinson's disease, immune mediated neuroinflammation including multiple sclerosis and neuromyelitis optica and cerebrospinal infections have inflammatory pathogenesis involving microgliosis and infiltration by macrophages, the administration of xanthohumol is expected to result in therapeutic inhibition of progression of all above diseases as well as of related neurodegenerative, immune mediated and infectious diseases of the brain and of the spinal cord.

### FIELD OF THE INVENTION

The present invention is about a therapeutic method where oral administration of xanthohumol improves clinical and pathologic outcomes in the spinal cord injury (SCI), in the traumatic brain injury (TBI), in the resection neurosurgery and in stroke, and also in cerebral edema and in spinal cord edema.

### BACKGROUND OF THE INVENTION

Treatments for neurotrauma and vascular occlusive accidents resulting in stroke are lacking or are generally not effective. This unfortunate status has not been helped by poor understanding of the pathogenesis of these diseases with futile therapeutic strategies aimed at wrong pathologic targets. Neurotrauma in the spinal cord injury (SCI), in the traumatic brain injury (TBI) and in neurosurgical resection that results in a locally massive necrosis and hemorrhage in the white matter, initiate a severe and extraordinarily protracted inflammation fueled by potently immunogenic damaged myelin. Although stroke is not initiated by a traumatic event but rather by an occlusive vascular accident in the brain, the resulting ischemia, locally massive necrosis and sometimes hemorrhages involving the white matter, initiate the same severe inflammatory response. The pathogenesis of neurotrauma and stroke has recently been elucidated in a systematic study on the rat model of SCI [Kwiecien et al, 2020a; Kwiecien, 2022], where macrophage-rich infiltration is directed at removing myelin-rich necrotic debris and red blood cells and also results in elevation of pro-inflammatory cytokines including IL-1b, IL-6 and IFN-g, and chemokines, and in damage to the spinal cord around the initial lesion. Since the human brain is rich in the white matter content and TBI, surgical resection and stroke often involve this myelin-rich tissue, the myelin-rich spinal cord is an appropriate model of the white matter injury in cerebral trauma and stroke. Rodent models of cerebral trauma and stroke involve primarily a gray matter injury since the white matter content in the brain of mice and rats is low. A gray matter injury initiates inflammation that is much less severe and self-limiting. Severe inflammation initiated by neurotrauma (SCI, TBI, neurosurgical resection) and stroke involving the white matter, is associated with damage to small and capillary blood vessels in the CNS around the inflammatory lesion leading to leaking of excess fluid that can overwhelm water management by astrocytic systems leading to cerebral edema or spinal cord edema [Kwiecien et al, 2021d, Mallon et al, 2021]. It is considered that neuroinflammation initiated by TBI and SCI can be more damaging to neurological outcome than neurotrauma itself [Patterson & Holahan, 2012; Wofford et al, 2019]. While stroke [Schroeter et al, 1977] or neurotrauma-initiated inflammation has been recognized in the animal models of SCI [Arnold & Hagg, 2011; Batchelor et al, 2008; Huang et al, 2009; Lee et al, 2015, 2018; Mietto et al, 2015; Taylor et al, 2014] and in TBI [Clausen et al, 2019; Elliot et al, 2011; Glushakova et al, 2014, 2018; Hazelton et al, 2018; Kempuraj et al, 2021; Morganti et al, 2016a, 2016b; Perez-Polo et al, 2013; Takamyia et al, 2007; Tatara et al, 2021; Treuttner et al, 2018; Witcher et al, 2021; Wofford et al, 2019; Zanier et al, 2016] and in human neurotrauma [Chaban et al, 2020; Frugier et al, 2010; Jenkins et al, 2018], with macrophages starting to infiltrate the site of injury by day 3 [Batchelor et al, 2008; Blight 1992; Gensel et al, 2015; Giulian & Robertson, 1990; Goldsmith et al, 2015; Hsieh et al, 2014; Kim et al, 2016; Kubo et al, 2000; Li et al, 2021; Luchetti et al, 2010; Nakajima et al, 2012; Norden et al, 2019; Popovich et al, 1997; Saber et al, 2017; Schwab et al, 2001; Takigawa et al, 2010; Wicher et al, 2017], the inflammatory disease has not been studied systematically along its entire course until recently [Kwiecien et al, 2020a]. Also, an anti-inflammatory and anti-edema tissue reaction in the spinal cord [Kwiecien 2013; Kwiecien et al, 2020a, 2021d] that appears to have a beneficial effect on the neurologic function [Kwiecien et al, 2019] and to inhibit and ultimately eliminate macrophages from the site of trauma [Kwiecien et al, 2020a] has not been previously addressed. The progressively severe astrocytic response to neurotrauma and stroke or astrogliosis is the most obvious cellular reaction associated with anti-inflammatory and anti-edema effect in the SCI [Kwiecien et al, 2020a, 2020d] and while molecular mechanisms playing role in these beneficial functions currently are unknown, their effect on the neurological scores and macrophage counts needs to be plotted in untreated animal models of neurotrauma and stroke against improvement related to experimental anti-inflammatory treatments until macrophages are eliminated from the site of injury [Kwiecien, 2021a]. This important parameter of preclinical studies on anti-inflammatory effect of experimental treatments has only recently been addressed by the author [Kwiecien et al, 2020b, Kwiecien, 2021a] but not by the others.

A cerebral vascular occlusion can cause ischemia and result in stroke. The resulting focal necrosis involving the white matter initiates severe, destructive, macrophage-rich inflammation not unlike that caused by SCI and TBI [Eldahshan et al, 2019; Jayaraj et al, 2019; Jian et al, 2019; Jin et al, 2010; Kanazawa et al, 2017; Kim et al, 2016; Lambertsen et al, 2012; Nakamura et al, 2019; Shekar et al, 2018; Zhao et al, 2017] with rodent models of stroke used to study and to treat it [Cai et al, 2019; Esposito et al, 2019; Wang et al, 2018; Zhang et al, 2019 a, 2019b; Zheng et al, 2019]. limiting cerebral and spinal edema [Kwiecien et al, 2021d]. Currently, there are no

While neuroinflammatory diseases discussed above are or are expected to be associated with vascular damage and cerebrospinal edema, this obvious relation has not been addressed until recently [Kwiecien et al, 2021d]. Anti-inflammatory agents therefore, are expected to inhibit vascular damage, lead to reduced fluid leakage thus satisfactorily effective treatments of inflammation at the core of pathogenesis of neurotrauma and stroke, thus effective anti-inflammatory treatments should address this shortfall limiting cerebral and spinal edema [Kwiecien et al, 2021d]. Currently, there are no

The inhibition and elimination of the severe inflammation initiated by neurotrauma and stroke is the first and necessary step in treatment of these diseases. By inhibiting inflammation, the inflammatory damage to cerebral and spinal cord blood vessels can be reduced and eliminated and the elimination of edema fluid accelerated. Based on the understanding of the pathogenesis of neurotrauma and stroke, anti-inflammatory agents have been infused subdurally in the vicinity of the SCI lesion in the rat model and the reduction of macrophage numbers in the lesion associated with improvement of neurologic deficits was observed [Kwiecien et al, 2015, 2016, 2019, 2020b]. Although inhibitory in 1-2 weeks studies, anti-inflammatory agents did not eliminate the inflammation and it took 8 weeks of a constant subdural infusion of an effective agent to reduce numbers of phagocytic macrophages to low levels observed in the untreated rats at 16 weeks post-SCI [Kwiecien et al, 2020a, 2020b]. The following parameters of a successful treatment to inhibit and eliminate the severe inflammation need to be considered in a neuroprotective therapy; (i) a potent anti-inflammatory agent that can reduce numbers of macrophages in the lesion; (ii) the route of administration effective in delivery of a candidate drug across or around the blood brain barrier (BBB) to the lesion. While the intralesional administration has proven effective via subdural infusion and also by delivery of an anti-inflammatory agent from an implanted hydrogel [Kwiecien et al, 2019, 2020b, 2020c] it involves invasive neurosurgery. A systemic administration, such as oral or intravenous, while much less invasive and more practical to administer, needs to take under consideration the effect of inflammatory damage to the BBB-and to the brain spinal cord barrier (BSCB) and a damage-counteracting effect of astroglial reaction directed at restoring the BBB and/or BSCB. (iii) The duration of sustained administration of an anti-inflammatory agent has to be sufficient to eliminate macrophages from the lesion [Kwiecien et al, 2020b, 2021a]. A successful anti-inflammatory treatment in neurotrauma and stroke will reduce the damage to the CNS around the initial traumatic, ischemic lesion and will accelerate elimination of edema leading to reduction in neurologic deficits and improved quality of life in acute patients, particularly those where the traumatic or ischemic injury is not extensive. In patients with large traumatic or stroke lesions, an effective anti-inflammatory treatment may not restore enough neuroloical function by itself for acceptable quality of life, but it will allow for the application of tissue engineering therapies leading to neuroregeneration and greater degree of restoration of neurologic function [Kwiecien, 2021b; Kwiecien, 2022]. Such neuroengineering therapies are not considered possible when the severe destructive inflammation is active. Novel methods to measure the anti-inflammatory activity of a candidate agent have been developed in the rat model of SCI.

While neuroinflammatory diseases discussed above are or are expected to be associated with vascular damage and cerebrospinal edema, this obvious relation has not been addressed until recently [Kwiecien et al, 2021d]. Anti-inflammatory agents therefore, are expected to inhibit vascular damage, lead to reduced fluid leakage thus satisfactorily effective treatments of inflammation at the core of pathogenesis of neurotrauma and stroke, thus effective anti-inflammatory treatments should address this shortfall limiting cerebral and spinal edema [Kwiecien et al, 2021d]. Currently, there are no The inhibition and elimination of the severe inflammation initiated by neurotrauma and stroke is the first and necessary step in treatment of these diseases. By inhibiting inflammation, the inflammatory damage to cerebral and spinal cord blood vessels can be reduced and eliminated and the elimination of edema fluid accelerated. Based on the understanding of the pathogenesis of neurotrauma and stroke, anti-inflammatory agents have been infused subdurally in the vicinity of the SCI lesion in the rat model and the reduction of macrophage numbers in the lesion associated with improvement of neurologic deficits was observed [Kwiecien et al, 2015, 2016, 2019, 2020b]. Although inhibitory in 1-2 weeks studies, anti-inflammatory agents did not eliminate the inflammation and it took 8 weeks of a constant subdural infusion of an effective agent to reduce numbers of phagocytic macrophages to low levels observed in the untreated rats at 16 weeks post-SCI [Kwiecien et al, 2020a, 2020b]. The following parameters of a successful treatment to inhibit and eliminate the severe inflammation need to be considered in a neuroprotective therapy; (i) a potent anti-inflammatory agent that can reduce numbers of macrophages in the lesion; (ii) the route of administration effective in delivery of a candidate drug across or around the blood brain barrier (BBB) to the lesion. While the intralesional administration has proven effective via subdural infusion and also by delivery of an anti-inflammatory agent from an implanted hydrogel [Kwiecien et al, 2019, 2020b, 2020c] it involves invasive neurosurgery. A systemic administration, such as oral or intravenous, while much less invasive and more practical to administer, needs to take under consideration the effect of inflammatory damage to the BBB-and to the brain spinal cord barrier (BSCB) and a damage-counteracting effect of astroglial reaction directed at restoring the BBB and/or BSCB. (iii) The duration of sustained administration of an anti-inflammatory agent has to be sufficient to eliminate macrophages from the lesion [Kwiecien et al, 2020b, 2021a]. A successful anti-inflammatory treatment in neurotrauma and stroke will reduce the damage to the CNS around the initial traumatic, ischemic lesion and will accelerate elimination of edema leading to reduction in neurologic deficits and improved quality of life in acute patients, particularly those where the traumatic or ischemic injury is not extensive. In patients with large traumatic or stroke lesions, an effective anti-inflammatory treatment may not restore enough neurological function by itself for acceptable quality of life, but it will allow for the application of tissue engineering therapies leading to neuroregeneration and greater degree of restoration of neurologic function [Kwiecien, 2021b; Kwiecien, 2022]. Such neuroengineering therapies are not considered possible when the severe destructive inflammation is active. Novel methods to measure the anti-inflammatory activity of a candidate agent have been developed in the rat model of SCI.

In the Hind End Locomotor test, performed once every day in unrestrained rats in the cage, the motor function is measured with movements of both hind legs scored 0-6 with 0=complete paralysis and 6=normal gait [Kwiecien et al, 2019, 2020b, 2020c].

In the Toe Pinch Withdrawal test, performed once every day in rats held by the tail, the sensory function and the strength of both hind limbs are scored 0-6 with 0=complete loss of reaction to the toe pinch and 6=strong, normal withdrawal in both legs after the toe pinch [Kwiecien et al, 2019, 2020b, 2020c].

The Body Weight Change after the SCI surgery is recorded every 3^{rd} day [Kwiecien et al, 2019,2020b 2021c]. The histologic examination is performed in the series of consecutive sections, 3 mm thick, of the spinal cord including the lesion, stained with luxol fast blue and counterstained with hematoxylin and eosin (LFB+H&E). A necrotic and hemorrhagic lesion deep in the spinal cord is infiltrated starting at the day 3, by numerous inflammatory, CD68+/CD163− macrophages that phagocytize myelin-rich necrotic debris and red blood cells. The numbers of macrophages rapidly increase and remain high for 4 weeks after SCI after which they gradually decline [Kwiecien et al, 2020a]. Within the first week post-SCI the deep lesion is transformed into a cavity of injury (COI) when it starts to accumulate excess water from edema in the surrounding spinal cord [Kwiecien et al, 2020a, 2021d]. The macrophage count is performed in a COI in the injured spinal cord of untreated and treated rats in a standardized fashion at fixed periods of time post-surgery to determine a degree of reduction of macrophage numbers related to a tested anti-inflammatory agent [Kwiecien et al, 2019; 2020a, 2020b, 2021c]. This reduction in numbers of macrophages plotted against such numbers in controls is reliably indicative of a therapeutic effect of an anti-inflammatory agent.

Xanthohumol (2′,4′,4-trihydroxy-6′-methoxy-3′-prenylchalcone) is a major chalcone derived from hops (Humulus lupulus L.) [Lee et al, 2011] isolated by stages of elution by organic solvents [Stevens & Page, 2004]. Total synthesis of xanthohumol has been described [Khupse & Erharddt, 2007]. In in vitro and in pre-clinical studies on mechanistic and therapeutic activities of xanthohumol indicate a vast array of beneficial activities in inflammatory, neoplastic, infectious and metabolic diseases [Doddapattar et al, 2013; Lima-Fontes et al, 2017; Miranda et al, 2016; Monteiro et al, 2008; Negrao et al, 2010, Nozawa et al, 2005; Yamaguchi et al, 2008]. The interest in xanthohumol as a therapeutic agent has led to a clinical trial phase 1 in healthy volunteers, approved by the Food and Drug Administration, USA, designed to test it for potential toxicity [Bradley et al, 2020]. Powerful anti-inflammatory activity of xanthohumol has been shown in animal models of hepatitis [Dorn et al, 2010a, 2013; Yang et al, 2013], pneumonia [Lv et al, 2017], cisplatin induced nephrotoxicity [Li et al, 2018], allergic dermatitis [Cho et al, 2010], skin wound healing in type 1 diabetes mellitus mice [Costa et al, 2013] and in normal rats [Negrao et al, 2010, 2012], osteoarthritis [Chen et al, 2021; Khayyal et al, 2020; Zhang et al, 2021], colitis [Cho et al, 2017], atherosclerotic plaque formation [Doddapattar et al, 2013], and attributed to inhibition of nuclear factor NF-kB [Chen et al, 2008; Cho et al, 2008; Dorn et al, 2010a], pro-inflammatory cytokines including IL-1b, TNF-a, IL-6, Il-12 [Cho et al, 2008, 2010] and other pro-inflammatory factors including iNOS [Cho et al, 2008]. In vitro studies have shown potent inhibition of pro-inflammatory macrophage activity by xanthohumol including reduction of IL-1b, TNF-a and macrophage chemoattractant protein-1 [Chen et al, 2008; Lupinnaci et al, 2009]. Anti-inflammatory and anti-oxidative activities of xanthohumol in mouse BV2 microglia were shown to be related to elevation of NRF2 protein and inhibition of nuclear factor NF-kB, IL-1b and TNF-a [Lee et al, 2011]. Xanthohumol has been shown to have neuroprotective activity in a mouse model of retinal degeneration [Henneman et al, 2018], in age-related inflammatory and apoptotic brain damage in male senescence-accelerated prone mice [Rancan et al, 2017], in ischemic rat model [Yen et al, 2012], in kainic acid excitotoxicity in rats [Wang et al, 2020] and also in an in vitro study where it also had a neuroprotective effect and promoted neuronogenesis and axonal growth [Oberbauer et al, 2013] suggesting the systemic administration of xanthohumol as potentially useful adjunctive agent in neuroregenerative therapies. In a rat model of stroke, xanthohumol, has been shown to limit the size of the lesion [Jiao et al, 2020; Yen et al, 2012], however, its anti-inflammatory activity in this model has not been demonstrated. The oral administration of xanthohumol has been shown to result in low bioavailability likely related to metabolic degradation by intestinal microflora [Legette et al, 2012, Nookandeh et al, 2004, Stevens & Page, 2004]. Since xanthohumol dissolves in the water poorly, addition of ethyl alcohol, cremophor, tween20, propylene glycol, its micellar solubilization or Hydroxypropyl-β-cyclodextrin complexing, to increase its solubility for topical, oral or parenteral administration have been attempted with considerable success [Henneman et al, 2018; Husson et al, 2005; Khayyal et al, 2020; Kirchinger et al, 2019; Legette et al, 2012.

While neuroinflammatory diseases discussed above are or are expected to be associated with vascular damage and cerebrospinal edema, this obvious relation has not been addressed until recently [Kwiecien et al, 2021d]. Anti-inflammatory agents therefore, are expected to inhibit vascular damage, lead to reduced fluid leakage thus limiting cerebral and spinal edema [Kwiecien et al, 2021d]. Currently, there are no satisfactorily effective treatments of inflammation at the core of pathogenesis of neurotrauma and stroke, thus effective anti-inflammatory treatments should address this shortfall.

The inhibition and elimination of the severe inflammation initiated by neurotrauma and stroke is the first and necessary step in treatment of these diseases. By inhibiting inflammation, the inflammatory damage to cerebral and spinal cord blood vessels can be reduced and eliminated and the elimination of edema fluid accelerated. Based on the understanding of the pathogenesis of neurotrauma and stroke, anti-inflammatory agents have been infused subdurally in the vicinity of the SCI lesion in the rat model and the reduction of macrophage numbers in the lesion associated with improvement of neurologic deficits was observed [Kwiecien et al, 2015, 2016, 2019, 2020b]. Although inhibitory in 1-2 weeks studies, anti-inflammatory agents did not eliminate the inflammation and it took 8 weeks of a constant subdural infusion of an effective agent to reduce numbers of phagocytic macrophages to low levels observed in the untreated rats at 16 weeks post-SCI [Kwiecien et al, 2020a, 2020b]. The following parameters of a successful treatment to inhibit and eliminate the severe inflammation need to be considered in a neuroprotective therapy; (i) a potent anti-inflammatory agent that can reduce numbers of macrophages in the lesion; (ii) the route of administration effective in delivery of a candidate drug across or around the blood brain barrier (BBB) to the lesion. While the intralesional administration has proven effective via subdural infusion and also by delivery of an anti-inflammatory agent from an implanted hydrogel [Kwiecien et al, 2019, 2020b, 2020c] it involves invasive neurosurgery. A systemic administration, such as oral or intravenous, while much less invasive and more practical to administer, needs to take under consideration the effect of inflammatory damage to the BBB-and to the brain spinal cord barrier (BSCB) and a damage-counteracting effect of astroglial reaction directed at restoring the BBB and/or BSCB. (iii) The duration of sustained administration of an anti-inflammatory agent has to be sufficient to eliminate macrophages from the lesion [Kwiecien et al, 2020b, 2021a]. A successful anti-inflammatory treatment in neurotrauma and stroke will reduce the damage to the CNS around the initial traumatic, ischemic lesion and will accelerate elimination of edema leading to reduction in neurologic deficits and improved quality of life in acute patients, particularly those where the traumatic or ischemic injury is not extensive. In patients with large traumatic or stroke lesions, an effective anti-inflammatory treatment may not restore enough neurological function by itself for acceptable quality of life, but it will allow for the application of tissue engineering therapies leading to neuroregeneration and greater degree of restoration of neurologic function [Kwiecien, 2021b; Kwiecien, 2022]. Such neuroengineering therapies are not considered possible when the severe destructive inflammation is active. Novel methods to measure the anti-inflammatory activity of a candidate agent have been developed in the rat model of SCI.

In the Hind End Locomotor test, performed once every day in unrestrained rats in the cage, the motor function is measured with movements of both hind legs scored 0-6 with 0=complete paralysis and 6=normal gait [Kwiecien et al, 2019, 2020b, 2020c].

In the Toe Pinch Withdrawal test, performed once every day in rats held by the tail, the sensory function and the strength of both hind limbs are scored 0-6 with 0=complete loss of reaction to the toe pinch and 6=strong, normal withdrawal in both legs after the toe pinch [Kwiecien et al, 2019, 2020b, 2020c].

The Body Weight Change after the SCI surgery is recorded every 3^{rd} day [Kwiecien et al, 2019,2020b 2021c].

The histologic examination is performed in the series of consecutive sections, 3 mm thick, of the spinal cord including the lesion, stained with luxol fast blue and counterstained with hematoxylin and eosin (LFB+H&E). A necrotic and hemorrhagic lesion deep in the spinal cord is infiltrated starting at the day 3, by numerous inflammatory, CD68+/CD163− macrophages that phagocytize myelin-rich necrotic debris and red blood cells. The numbers of macrophages rapidly increase and remain high for 4 weeks after SCI after which they gradually decline [Kwiecien et al, 2020a]. Within the first week post-SCI the deep lesion is transformed into a cavity of injury (COI) when it starts to accumulate excess water from edema in the surrounding spinal cord [Kwiecien et al, 2020a, 2021d]. The macrophage count is performed in a COI in the injured spinal cord of untreated and treated rats in a standardized fashion at fixed periods of time post-surgery to determine a degree of reduction of macrophage numbers related to a tested anti-inflammatory agent [Kwiecien et al, 2019; 2020a, 2020b, 2021c]. This reduction in numbers of macrophages plotted against such numbers in controls is reliably indicative of a therapeutic effect of an anti-inflammatory agent.

Xanthohumol (2′,4′,4-trihydroxy-6′-methoxy-3′-prenylchalcone) is a major chalcone derived from hops (Humulus lupulus L.) [Lee et al, 2011] isolated by stages of elution by organic solvents [Stevens & Page, 2004]. Total synthesis of xanthohumol has been described [Khupse & Erharddt, 2007]. In in vitro and in pre-clinical studies on mechanistic and therapeutic activities of xanthohumol indicate a vast array of beneficial activities in inflammatory, neoplastic, infectious and metabolic diseases [Doddapattar et al, 2013; Lima-Fontes et al, 2017; Miranda et al, 2016; Monteiro et al, 2008; Negrao et al, 2010, Nozawa et al, 2005; Yamaguchi et al, 2008]. The interest in xanthohumol as a therapeutic agent has led to a clinical trial phase 1 in healthy volunteers, approved by the Food and Drug Administration, USA, designed to test it for potential toxicity [Bradley et al, 2020]. Powerful anti-inflammatory activity of xanthohumol has been shown in animal models of hepatitis [Dorn et al, 2010a, 2013; Yang et al, 2013], pneumonia [Lv et al, 2017], cisplatin induced nephrotoxicity [Li et al, 2018], allergic dermatitis [Cho et al, 2010], skin wound healing in type 1 diabetes mellitus mice [Costa et al, 2013] and in normal rats [Negrao et al, 2010, 2012], osteoarthritis [Chen et al, 2021; Khayyal et al, 2020; Zhang et al, 2021], colitis [Cho et al, 2017], atherosclerotic plaque formation [Doddapattar et al, 2013], and attributed to inhibition of nuclear factor NF-kB [Chen et al, 2008; Cho et al, 2008; Dorn et al, 2010a], pro-inflammatory cytokines including IL-1b, TNF-a, IL-6, Il-12 [Cho et al, 2008, 2010] and other pro-inflammatory factors including iNOS [Cho et al, 2008]. In vitro studies have shown potent inhibition of pro-inflammatory macrophage activity by xanthohumol including reduction of IL-1b, TNF-a and macrophage chemoattractant protein-1 [Chen et al, 2008; Lupinnaci et al, 2009]. Anti-inflammatory and anti-oxidative activities of xanthohumol in mouse BV2 microglia were shown to be related to elevation of NRF2 protein and inhibition of nuclear factor NF-kB, IL-1b and TNF-a [Lee et al, 2011]. Xanthohumol has been shown to have neuroprotective activity in a mouse model of retinal degeneration [Henneman et al, 2018], in age-related inflammatory and apoptotic brain damage in male senescence-accelerated prone mice [Rancan et al, 2017], in ischemic rat model [Yen et al, 2012], in kainic acid excitotoxicity in rats [Wang et al, 2020] and also in an in vitro study where it also had a neuroprotective effect and promoted neuronogenesis and axonal growth [Oberbauer et al, 2013] suggesting the systemic administration of xanthohumol as potentially useful adjunctive agent in neuroregenerative therapies. In a rat model of stroke, xanthohumol, has been shown to limit the size of the lesion [Jiao et al, 2020; Yen et al, 2012], however, its anti-inflammatory activity in this model has not been demonstrated. The oral administration of xanthohumol has been shown to result in low bioavailability likely related to metabolic degradation by intestinal microflora [Legette et al, 2012, Nookandeh et al, 2004, Stevens & Page, 2004]. Since xanthohumol dissolves in the water poorly, addition of ethyl alcohol, cremophor, tween20, propylene glycol, its micellar solubilization or Hydroxypropyl-β-cyclodextrin complexing, to increase its solubility for topical, oral or parenteral administration have been attempted with considerable success [Henneman et al, 2018; Husson et al, 2005; Khayyal et al, 2020; Kirchinger et al, 2019; Legette et al, 2012].

While the oral administration of XN results in poor bioavailability, approximately 1%, the amount of XN in blood plasma appears to be elevated with increasing oral dosing in mice, rats and humans [Legette et al., 2012; Neumann et al., 2022]. There is a direct therapeutic consequence of this since increasing oral doses of XN resulted in increasingly better scores of neurological deficits with their faster recovery coinciding with remarkable inhibition of macrophage counts in the cavity of injury (COI) combined with accelerated elimination of macrophages from the COI [Kwiecien, 2022]. The most comprehensive study on pharmacology of hops XN in Wistar rats [Nowak et al, 2020] indicates that oral administration of 40 to 200 mg/kg results in declining bioavailability corresponding to 1.16 to 0.53% respectively. The half-life of 10 mg/kg XN administered intravenously is 7.67 hours while it is 21 to 23 hours after oral administration of 40 to 200 mg/kg [Nowak et al, 2022]. Relatively short duration of xanthohumol bioavailability in blood indicates a need for multiple oral administrations per day or the continuous parenteral administration to maintain its optimal therapeutic level. Specific studies on rats and mice revealed no toxicity after long administration of large doses of xanthohumol [Dorn et al, 2010b; Husson et al, 2005; Vanhoecke et al, 2005].

### BRIEF SUMMARY OF THE INVENTION

A method to treat the spinal cord injury (SCI), traumatic brain injury (TBI), neurosurgical resection, stroke and cerebral edema and spinal cord edema by the oral administration of 5 mg xanthohumol or similar per day for a period of 8 weeks or similar is described in the rat model of SCI. This treatment approximates 15 mg/kg body weight and has the anti-inflammatory effect and causes neuroprotection in the SCI rats with improvement of neurologic functions and other clinical signs. The neurologic function was measured by novel methodology designed to broaden examination of a variety of functions improving after the SCI and to increase its robustness; the Hind End Locomotor Function test, the Toe Pinch Withdrawal test and the Duration of Paralysis of the Urinary Bladder. The body weight measurements were taken as well. While phagocytic macrophages are the main cellular effector in a severe, destructive and long-lasting inflammation, the Macrophage Count in the Cavity of Injury (COI) was performed. The administration of xanthohumol was performed twice a day and this frequency and the dosing itself can be increased since toxicity of xanthohumol was not observed. The improvements in neurologic deficits and in accelerated reduction and elimination of phagocytic macrophages on histologic examination indicate a reduced inflammatory damage to the spinal cord surrounding the lesion and also, indicate an accelerated reduction and elimination of spinal cord edema therein. The treatment with xanthohumol presented here is novel, effective and the first and necessary anti-inflammatory treatment in neurotrauma including the SCI, TBI, and the neurosurgical resection and in stroke.

### BRIEF DESCRIPTION OF THE DRAWINGS

FIG. 1. Shows the results of the Hind End Locomotor test was performed on rats in groups treated with xanthohumol, surviving for 1-8 weeks post-SCI. Analysis of Variance (ANOVA) was performed to obtain an overall F-value for the model as well as individual T-values. At the same time p-values were produced for the overall model and individual variables. This was done for 1 week, 2 week, 4 week, 6 week, and 8 week study groups. The effect of the treatment was statistically significant for each group with duration 2-8 weeks post-SCI.

FIG. 2. Shows the results of the Toe Pinch Withdrawal test in rats treated with xanthohumol, for 1-8 weeks post-SCI. The analysis of variance (ANOVA) was performed, p-values were produced for the overall model and individual variables. The effect of the treatment was statistically significant for groups with duration 2-8 weeks post-SCI.

FIG. 3. Shows the Duration of Paralysis of the Urinary Bladder performed in rats treated with xanthohumol for 1-8 weeks post-SCI. The Analysis of Variance (ANOVA) was performed and p-values were produced. The effect of the treatment was statistically significant for the 4 weeks post-SCI group but not for other groups although there was the tendency for xanthohumol rats to recover bladder function faster in the 6 weeks post-SCI group

FIG. 4. Shows the Body Weight Change in rats every 3rd day and expressed as average of percentage of the body weight taken before the surgery. The Analysis of Variance (ANOVA) was performed to obtain p-values. While rats treated with xanthohumol for 4 and for 6 weeks recovered the body weight faster and better than untreated controls, there was no statistical difference between both groups of rats in 1, 2 and 8 weeks post-SCI

FIG. 5. Shows the dose-response to treatment with xanthohumol analysed in the Macrophage Counts in the Cavity of Injury (COI) in SCI rats. All 3 treatment doses of xanthohumol had a macrophage-lowering effect with a clear dose-response effect which was statistically significant for 1.0 and 5.0 mg/rat/day but not for 0.2 mg mg/rat/day.

FIG. 6. Shows the reducing effect of 5 mg/rat/day of xanthohumol on macrophage counts and anti-inflammatory effect in the rat model of SCI. The macrophage counts in the COI, were gradually reduced from a highest at 1 week to the lowest at 8 weeks post-SCI in untreated rats and more so in xanthohumol-treated rats. The triangle-shaped black markers indicate average % macrophage counts of xanthohumol treated rats vs untreated controls and these percentages are indicated at each time point from 1-8 weeks post-SCI. While the averages of the macrophage counts were lower in xanthohumol-treated rats at each time point post-SCI, they were statistically different for groups 1-6 weeks post-SCI but not for the group analysed at 8 weeks. The Analysis of Variance (ANOVA) was performed to obtain p-values. This was done for 1 week, 2 week, 4 week, 6 week, and 8 week study groups. Another ANOVA was performed incorporating time in days as a second independent variable.

FIG. 7. Shows color microphotographs with histologic representation of the SCI in the rat model at 1-8 weeks post-SCI. The sections of the spinal cord contain a traumatic lesion converted into a cavity of injury (COI) indicated by a yellow star and delineated by the surrounding spinal cord (yellow arrowheads). The COI is infiltrated by macrophages (indicated by an open yellow arrow); large cells with a round or oval nucleus and large cytoplasm containing blue granules of myelin and/or red blood cells. The greatest numbers of macrophages are at 1 week post-SCI and then gradually decline at weeks 2-8 in untreated rats (column 2). In xanthohumol-treated rats numbers of macrophages in the COI are reduced at each time point post-SCI (column 4) and are rare or absent at 6 and 8 weeks post-SCI. Size bars; 1 mm for micrographs in columns 1 and 3; 50 μm for micrographs in columns 2 and 4. Luxol fast blue counterstained with hematoxylin and eosin (LFB+H&E).

FIG. 8. This graph shows the macrophage count over time in SCI rats treated with xanthohumol obtained from hops or synthetic xanthohumol.

FIG. 9. These graphs show the optimal macrophage-reducing dosage of A) Hop-xanthohumol and B) synthetic xanthohumol.

### DETAILED DESCRIPTION OF THE INVENTION

The present invention relates to the systemic administration of xanthohumol to inhibit inflammation in the central nervous system to provide a neuroprotective effect and anti-edema effect. Thus, the administration of xanthohumol to a mammal is useful to treat neuroinflammatory diseases and conditions such as spinal cord injury, traumatic brain injury, neurosurgical resection, and stroke resulting in better neurological outcomes.

In an aspect of the invention, a method of reducing the number of macrophage cells at a cavity of injury in a central nervous system of a mammal is provided. The method comprises the step of administering a treatment dose of xanthohumol to the mammal.

Xanthohumol for use in the present methods may be naturally-occurring xanthohumol obtained from plants, e.g. hops plant, or synthetic xanthohumol prepared using chemical synthetic techniques.

In one embodiment, synthetic xanthohumol may be synthesized from phloracetophenone which is prenylated using a Mitsunobu reaction to create a prenyl ether precursor, which then undergoes a para-Claisen rearrangement to introduce the prenyl group onto the aryl ring. A Claisen-Schmidt condensation is used to provide the main chalcone structure. Methoxymethyl protecting groups are utilized and removed under specific acidic conditions to prevent unwanted cyclization into isoxanthohumol.

Xanthohumol may alternatively be synthesized from naringenin, a relatively inexpensive flavonoid, through acylation, rearrangement, and further substitution.

Other chemical synthetic routes may also be used.

For use to treat neuroinflammation, xanthohumol, either naturally occurring or synthetically synthesized, is administered systemically to a mammal, including human and non-human mammals. Systemic administration may include enteral administration, i.e. via the gastrointestinal tract, for example orally, or parenterally, i.e. via methods that bypass the digestive system such as intravenous, intramuscular and subcutaneous injections. In one embodiment, xanthohumol is administered orally.

Xanthohumol is administered in a therapeutically effective amount, i.e. an amount which is effective to reduce neuroinflammation as evidenced by lower macrophage counts at a cavity of injury. Effective daily dosages of xanthohumol will vary on the mammal to be treated, the condition to be treated, the source of xanthohumol and the like, as will be appreciated by one of skill in the art. In embodiments, dosages in the range of 0.001-100 mg/kg per day may be used. For humans, daily dosages in the range of about 0.01-100 mg/kg per day, such as 0.5-50 mg/kg per day, 1-20 mg/kg per day or 1.5-15 mg/kg per day, including daily dosages of 5 mg/kg, 6 mg/kg, 7 mg/kg, 8 mg/kg, 9 mg/kg, 10 mg/kg, 11 mg/kg, 12 mg/kg, 13 mg/kg, 14 mg/kg and 15 mg/kg per day may be administered to treat neuroinflammation.

Daily dosages may be administered as a single daily dosage or may be administered as 2 or more dosages given throughout the day. Other treatment regimens may also be suitable including administration of dosages on a less frequent basis, such as every other day, or administration of variable dosages over time depending on the mammal's response to treatment.

Xanthohumol treatment is administered for a sufficient period of time to treat the neuroinflammation. In embodiments, the treatment may be for days, 1 or more weeks, or longer depending on the severity of the condition. The terms “treatment”, “treating” or “treat” are used herein to refer to curing the disease or condition, alleviation or amelioration of one or more symptoms or conditions, arresting development of disease, delay or slowing of disease progression, amelioration or palliation of the disease state, or prolonging of survival as compared to no treatment. Treatment may more specifically refer to reduction of macrophage count within a cavity of injury, reduction of inflammation at the cavity of injury, improvement (e.g. an increase) in motor function or improvement of other physical characteristics which deteriorated due to the disease or condition.

Neuroinflammatory conditions to be treated include, but are not limited to, spinal cord injury, traumatic brain injury, neurosurgical resection, and stroke, as well as diseases in which neuroinflammation plays a role such as neuromyelitis optica, multiple sclerosis, Amyotrophic Lateral Sclerosis, Parkinson's disease, frontotemporal dementia and Alzheimer's disease.

While not wishing to be bound by any particular theory, these conditions result in a macrophage reaction including migration of macrophages from the blood stream and infiltration into a cavity of injury. Numbers of infiltrating macrophages in the COI is much higher per area unit than macrophages in an extraneural soft tissue trauma or infection. Macrophages in the COI are exclusively CD68+/CD163− a pro-inflammatory type throughout 3-112 days post-SCI. When administered orally or parenterally, xanthohumol crosses the blood-spinal cord barrier and targets macrophages in the COI, thereby lowering macrophage numbers in the COI to exert its anti-inflammatory effect.

Embodiments of the invention are described in the following non-limiting examples.

### EXAMPLE 1

The spinal cord injury model in the rat.

Healthy male Long Evans rats aged 12 weeks, 330-360 g, were offered a fruit flavored jello cube twice a day for 1 week prior to the surgery and were separated in individual cages 3 days before the surgery. Rats were induced with 5% isoflurane in 95% oxygen flowing at a rate of 1 liter per minute and maintained at 3.5% isoflurane in 96.5% oxygen. The anaesthetized rats had the skin on the back shaved and prepared for surgery with 70% ethyl alcohol and 10% iodine swabs. The skin was cut over the caudal thoracic and lumbar spine and spinal muscles dissected from the vertebral spine of the thoracic 10 (T10) vertebrum and the dorsal arches of this vertebrum removed. A 3Fogarty catheter was inserted via this laminectomy over the intact dura towards the head to place the caudal edge of the 3 mm long balloon at 1 cm rostral to the laminectomy. The balloon was inflated with 15 μL of sterile saline for 3 minutes, then deflated and the catheter removed. The spinal muscles were closed with absorbable sutures over the laminectomy and the skin incision was closed with stainless steel staples. Before awakening, the rats were administered; 50 μL of a painkiller Anafen (ketoprofen, 100 mg/mL, Merial) subcutaneously for pain, and 50 μL of Baytril antibiotic (enrofloxacin 50 mg/mL, Bayer) intramuscular, and 3 mL of saline subcutaneous. In the initial experiment designed to determine the anti-inflammatory effect of 0.2, 1.0 and 5 mg per day of xanthohumol (Sigma-Aldrich) after the surgery, the rats were offered a jello cube with half of the above dose twice a day, at 8-9 hours and 15-16 hours intervals, and the consumption of the jello cube was observed and recorded every day for 7 days. Each treatment group had 5 rats. In the second phase of the experiment 1 jello cube alone or jello cube with 2.5 mg xanthohumol was offered twice a day for 1, 2, 4, 6, and 8 weeks. Each treatment group had 6-7 rats.

Given the invasive nature of the SCI model, an ethical Endpoint was instituted. A rat with distended urinary bladder that was impossible to express or was ruptured, or with severe dehydration and with hypothermia and lethargy was humanely euthanized and not used in the study. The rats were administered Anafen painkiller once daily for 2 days post-surgery and rats with distended urinary bladder were given Baytril antibiotic once daily for 5 days post-surgery. Rats with moderate dehydration were administered with 5-10 mL saline subcutaneously once or twice a day as needed. Rats with distended urinary bladder and micturition resulting in soiling of the perineal area had the bladder gently manually expressed twice a day and had a bath of the hind end in warm clean water done every 2 days until the bladder function returned.

The clinical tests:
1. The Hind End Locomotor test [Kwiecien et al, 2021c] was performed in freely moving rats in the cage once a day. The motor function of the hind limbs was scored from 0-6 where 0=complete paralysis of both hind legs and 6=normal locomotion.
2. The Toe Pinch Withdrawal test [Kwiecien et al, 2021c] was performed once a day in rats held by the tail in the cage and the presence and strength of both hind legs scored 0-6 where 0=no response to the toe pinch and 6=strong, normal withdrawal response to the toe pinch in both legs.
3. The balloon crush SCI and spinal bone and muscle trauma related to the surgery resulted in a body weight loss that was transient and recovered over a period of a few weeks. The rats were weighed every 3^{rd} day.
4. The duration of the distention of the urinary bladder that required manual voiding until the normal function of the urinary bladder was restored, in days, was recorded.

At a required experimental time point; 1, 2, 4, 6, or 8 weeks post-surgery, the SCI rats were overdosed with the sodium pentobarbital (80 mg/kg b.w.) administered intraperitoneally. When in deep plane of anaesthesia, the chest was cut open, 100 international units of heparin sodium injected into the left heart ventricle and a cannula with flowing lactated Ringer's solution inserted into the left ventricle while the right auricle was cut open. After the blood was washed out, the flow of the lactated Ringer's solution was replaced by that of phosphate buffered formalin and the carcass was fixed. The spine was removed, postfixed in formalin overnight and then placed in formalin supplemented with 8% EDTA, pH 7.0, to decalcify the spinal vertebrae. The decalcifying solution was replaced by fresh one every 2 days for 2 weeks. Once soft, the spine was cut perpendicular to its long axis into 3 mm thick segments starting from laminectomy rostrally to include the SCI lesion. Eight segments were processed in rising concentrations of ethyl alcohol and xylene, embedded in paraffin wax, cut 5 μm thick and mounted on the glass slide. The sections were stained with luxol fast blue and counterstained with hematoxylin and eosin (LFB+H&E) and coverslipped. Stained sections were examined by the experienced experimental neuropathologist (the author) under a Nikon Eclipse 50i light microscope and the spinal cord photographed. At 40× magnification a margin of one COI per section, including 20% of the spinal cord and 80% of the COI was digitally photographed. The 40× magnification images were then analyzed and macrophages; large cells with a round, oval, sometimes subcleaved nucleus with abundant cytoplasm containing blue granules of myelin debris and/or red blood cells [Kwiecien et al, 2015, 2016, 2019, 2020a, 2020b, 2021c], were counted. The counts were averaged for a rat and these averages averaged for a treatment group.

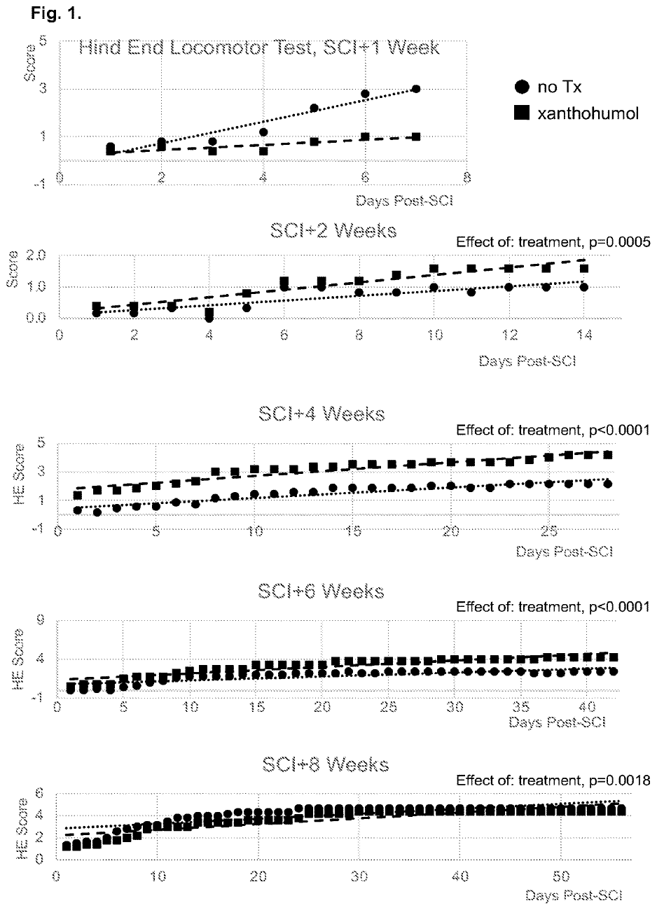

Results of this study on the anti-inflammatory effect of xanthohumol administered orally to rats with the SCI are demonstrated in FIGS. 1-7.

Details of statistical analysis of clinical data and of macrophage count data are provided respectively in the Brief Description of Drawings for FIGS. 1-6.

The Hind End Locomotor test (FIG. 1) revealed significantly higher scores for xanthohumol-treated rats vs untreated rats in treatment groups 2-8 weeks post-SCI, indicating beneficial effect of the treatment on a faster and stronger recovery of the motor function in the hind legs of SCI rats.

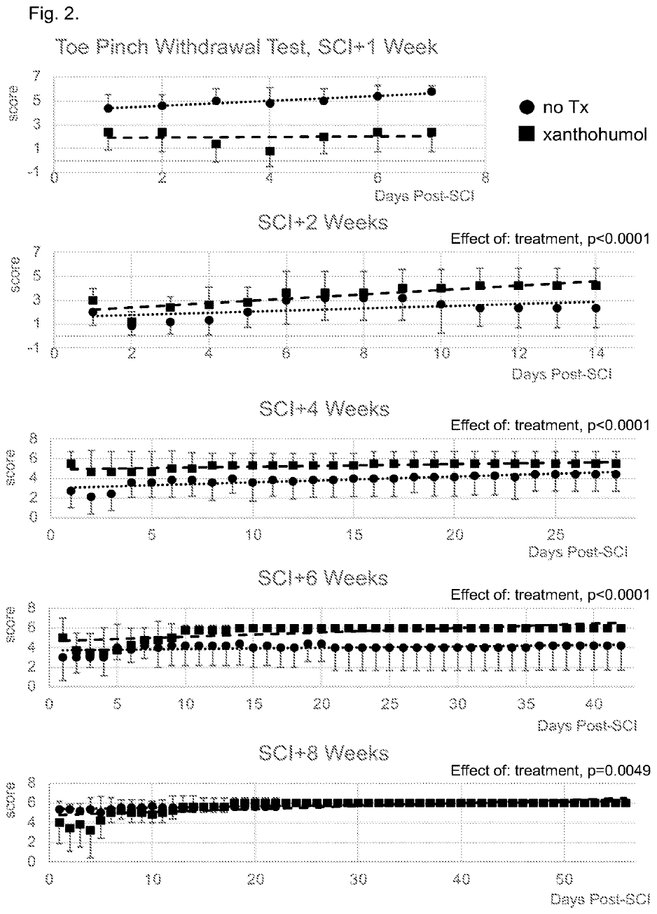

In the Toe Pinch Withdrawal test (FIG. 2), xanthohumol-treated SCI rats performed statistically better than untreated rats in treatment groups 2-8 weeks post-SCI, indicating beneficial effect of the treatment on the return of pain sensation and of withdrawal strength in the hind limbs.

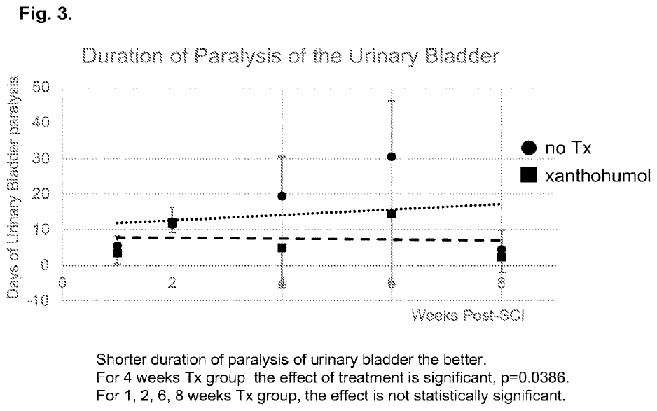

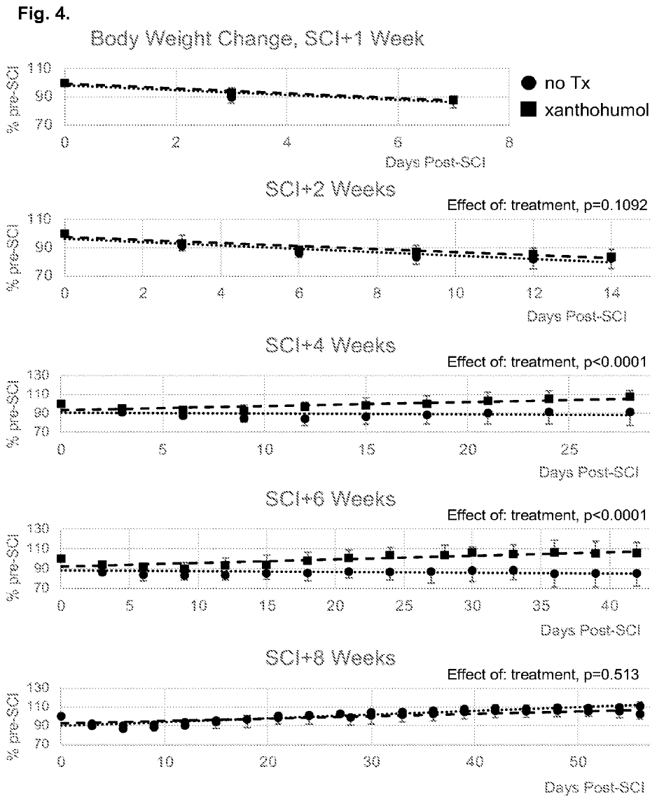

The duration of the distention of the urinary bladder due to its paralysis, that required manual voiding until the normal function of the urinary bladder was restored, is shown in FIG. 3. In the rats treated with xanthohumol recovery of the bladder function occurred significantly faster than in untreated rats in 4 weeks treatment group but not in other treatment groups, although in the 6 weeks treatment group there was a tendency for the paralyzed urinary bladder to recover faster than in controls. This novel previously not used test may indicate that the faster recovery of the urinary bladder function may be related to anti-inflammatory effect of xanthohumol and to related reduction to vascular damage and leaking of edema fluid in the spinal cord around and caudal to the lesion. Similar anti-inflammatory and edema-inhibiting mechanism may be of therapeutic benefit to the recovery of the breathing function, of the bowel function and to sexual performance. The body weight loss post-surgery and its recovery are presented in FIG. 4. While the body weight loss occurred in un-treated and in xanthohumol-treated SCI rats for the first 2 weeks, it recovered statistically sooner and to a greater degree in xanthohumol-treated rats than in untreated controls in groups treated for 4 and 6 weeks. This observation indicates that negative effects of the post-SCI inflammation included lower food intake in un-treated rats than in xanthohumol-treated rats.

The dose of xanthohumol of 5 mg per day was selected after performing a 7 days study on the SCI rats offered jello cubes with 0, 0.2, 1.0 and 5.0 mg of this agent. The

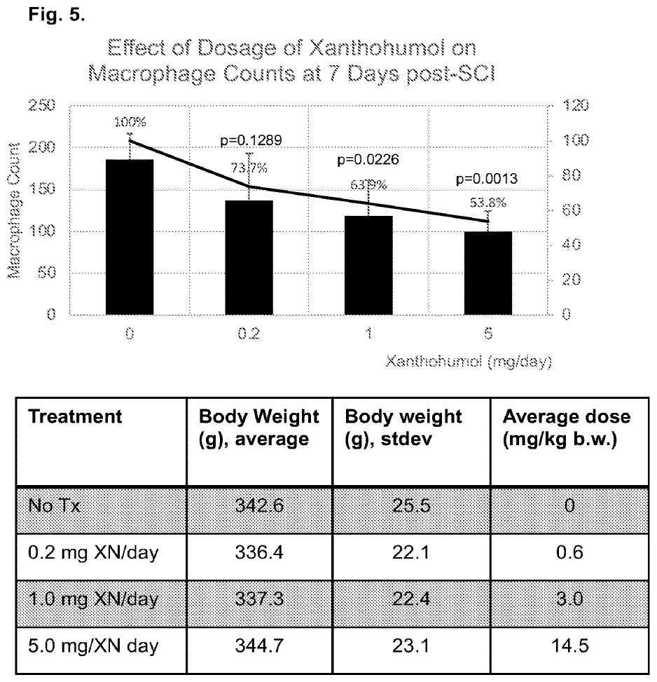

Macrophage Count in the COI test revealed that there was a dose response reduction in macrophage numbers at 73.7, 63.9, and 53.8% of the untreated controls (100%) (FIG. 5). The dosage of xanthohumol was calculated per kg body weight of average weight of treated rats before the surgery and it was 0.6, 3.0 and 14.5 g respectively (see Table in FIG. 5). The macrophage-reducing effect was caused by all 3 doses of xanthohumol, but this effect was dose-dependent and significant for 1.0 mg/rat/day (p=0.026) and for 5.0 mg/rat/day (p=0.0013). A 16.9 mg/kg body weight dose of daily oral administration of xanthohumol in rats revealed no toxic effect [Leggete et al, 2012] therefore, 5 mg of xanthohumol was offered in two 2.5 mg daily doses in the fruit jello to SCI rats in post-SCI studies conducted for 1, 2, 4, 6, and 8 weeks. This dose averaged 14.5 mg/kg body weight in rats immediately before SCI (see FIG. 5) and became higher than 14.5 mg/kg in rats post-SCI due to the body weight-lowering effect of this surgery (see FIG. 4).

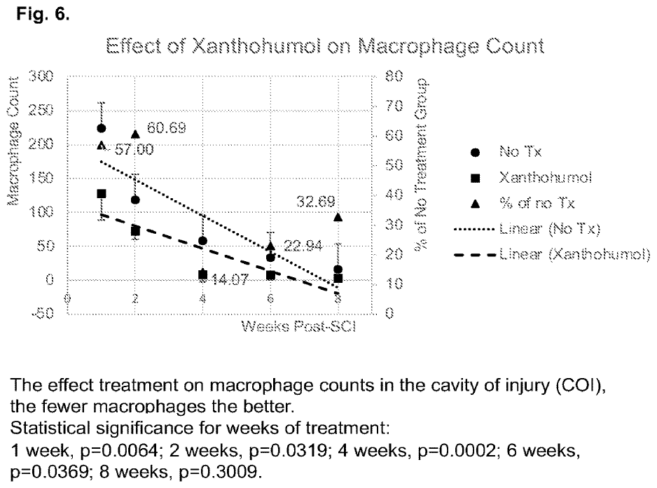

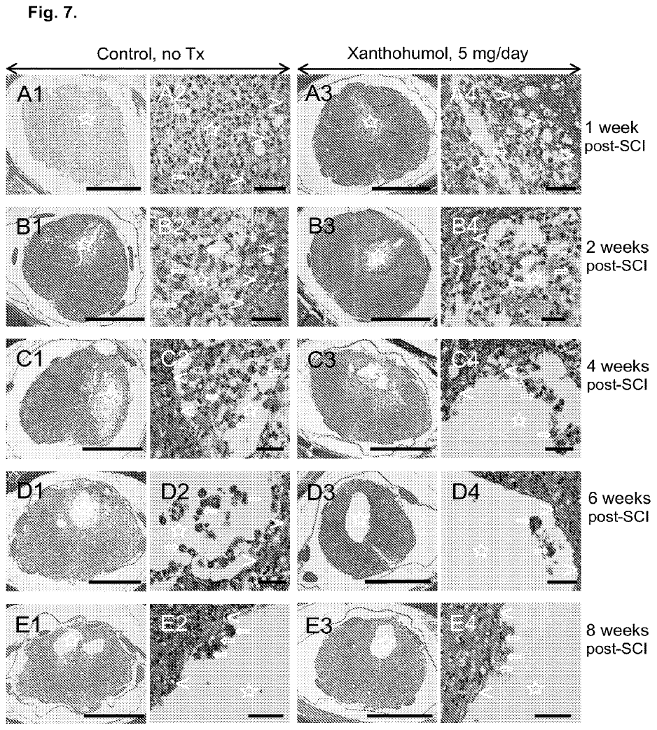

The results of the macrophage counts in un-treated rats and in rats treated with 5 mg xanthohumol per day are presented in the FIG. 6. While the numbers of macrophages in the COI in un-treated rats gradually declined after the 1st week due to an anti-inflammatory response by the spinal cord [Kwiecien et al, 2020a], the treatment with xanthohumol consistently accelerated reduction of these numbers at each study time point with statistical difference for 1-6 weeks treatment groups and not for 8 weeks treatment group. The macrophage counts in xanthohumol treated rats were lower at; 57.0, 60.7, 14.1, 22.9, and 32.7% of the controls respective to 1, 2, 4, 6, and 8 weeks post-SCI. These results indicate a powerful anti-inflammatory effect of orally administered xanthohumol resulting in reduction and accelerated elimination of phagocytic macrophages from the lesion initiated by the spinal cord injury. Since CD68+/CD163− pro-inflammatory macrophages persist in the COI and continue to destroy myelin in untreated rats beyond 16 weeks post-SCI [Kwiecien et al, 2020a], the anti-inflammatory effect of xanthohumol treatment evidenced as lowering numbers of phagocytic macrophages and accelerating their elimination, can be interpreted as neuroprotective in the SCI and also in the TBI and in stroke. The morphologic results of histologic analysis of the COI un-treated and xanthohumol-treated rats at 1, 2, 4, 6, and 8 weeks post-SCI are presented in FIG. 7.

### EXAMPLE 2

Xanthohumol (Hop-RXn) has also been used to treat neuroinflammation in human patients, and in particular, to treat patients with a spinal cord injury. The human dose was calculated to be about 60 mg for a patient weighing 65 kg as per the FDA recommendation using 6.2 coefficient to convert the dose used in rat to a human dose, taking into consideration differences in the body weight between rat and human.

A dose 60 mg Hop-RXn was administered 3× per day (180 mg/day per 65 kg body weight) in 9 SCI patients. At day 10 post-SCI, all patients treated with Hop-RXN:
i) had no edema on the cervical spinal cord at the vertebral fracture (reduced and stabilized surgically on day 1), as determined by MRI;
ii) showed recovery of motor function including: resumption of autonomous breathing allowing for the external respirator to be detached and lifting arms and legs in patients quadriplegic at the time of admission to the hospital; and
iii) showed reduced inflammation in the SCI lesion, i.e. COI measured as reduction of macrophages in the rat model. Thus, less severe inflammation allowed for efficient elimination of excess water from the spinal cord around the lesion with inflammation and recovery of motor function.

### EXAMPLE 3

The effect of xanthohumol obtained from Hops (Hop-RXn) was compared to the effect of synthetic xanthohumol (stXN) on macrophage-lowering effect in the spinal cord in SCI rats as described in Example 1.

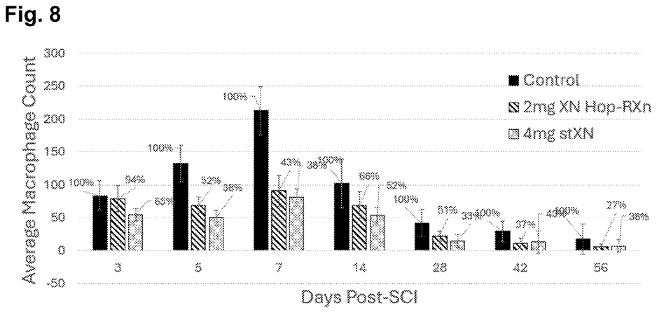

A macrophage-lowering effect in the spinal cord was observed when either xanthohumol obtained from Hops (Hop-RXn) or synthetic xanthohumol (stXN). However, markedly reduced numbers of macrophages and accelerated elimination of macrophages from the cavity of injury resulted when stXN was used. Significantly, as shown in FIG. 8, the macrophage-lowering effect of stXN started at day 3 while that of Hop-RXn started at day 5. The effect of stXN was consistently greater than that of Hop-RXn throughout the first 28 days and the effect of both treatments was similar during the second month when numbers of macrophages were low.

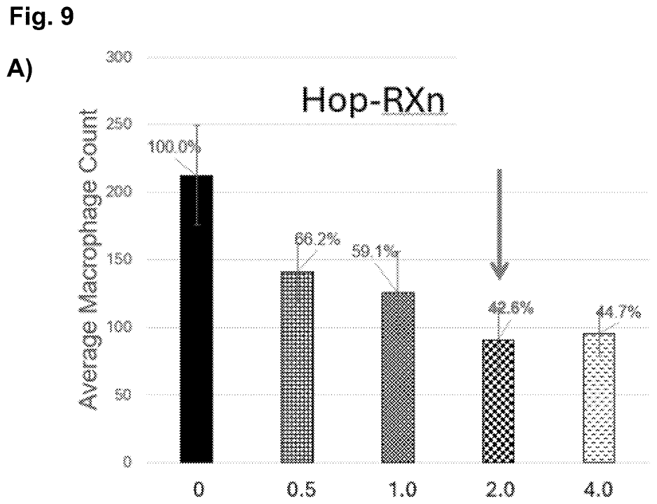

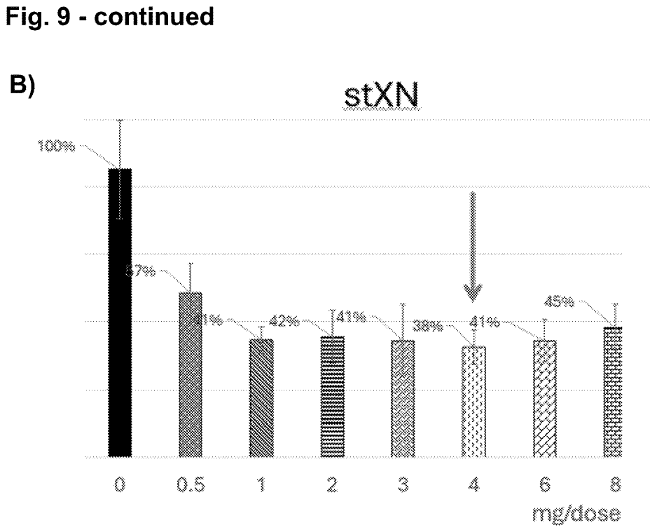

In addition, as shown in FIG. 9, in a study to determine the optimal dose for Hop-RXn and for stXN, the macrophage-lowering effect of increasing oral doses of both agents was tested in the rat model of SCI and measured with the macrophage count in the cavity of injury (COPI) test. While the optimal dose for Hop-RXn was determined to be 2 mg and that for stXN was determined to be 4 mg, the macrophage lowering effect in the long study: 3-56 days, demonstrated that the effect by stXN was superior.

These results have the following implications: (1) the effect of stXN is consistently better in the first 28 days than that of Hop-RXn, (2) the effect of stXN starts on day 3 vs day 5 for Hop-RXn. This means that the severity of inflammation is inhibited as soon as it starts, at day 3 post-SCI, (3) better inhibition of inflammation by stXN implies better neuroprotective effect=more spinal cord preserved, and (4) better inhibition of inflammation by stXN means faster elimination of vasogenic edema and recovery of motor function.

Thus, the anti-inflammatory and neuroprotective effect of stXN is superior to that of Hop-RXn.

1. A method of reducing the number of macrophage cells at a cavity of injury in a central nervous system in a mammal, the method comprising systemic administration to the mammal of a treatment dose of xanthohumol of 0.01-100 mg per kg body weight per day.

2. The method of claim 1, wherein neuroinflammation is inhibited in the mammal.

3. The method of claim 2, wherein the neuroinflammation is in spinal cord.

4. The method of claim 1, wherein the mammal has a spinal cord injury (SCI).

5. The method of claim 1, wherein the mammal is a human.

6. The method of claim 1, wherein the treatment dose of xanthohumol is a dosage of 0.5-50 mg/kg per day.

7. The method of claim 6, wherein the treatment dose of xanthohumol is a dosage of 1-20 mg/kg per day.

8. The method of claim 1, wherein the xanthohumol is naturally occurring.

9. The method of claim 1, wherein the xanthohumol is synthetic.

10. The method of claim 1, wherein the xanthohumol reduced macrophage number at the cavity of injury by at least about 20-40% within a week.

11. The method of claim 1, which additionally inhibits edema.

12. The method of claim 1, wherein the mammal has traumatic brain injury (TBI).

13. The method of claim 1, wherein the mammal has had a stroke.

14. The method of claim 1, wherein the mammal exhibits improved locomotor function as compared to an untreated control.

15. The method of claim 1, wherein the mammal exhibits a reduction in time of paralysis as compared to an untreated control.
